(19) United States
(12) Patent Application Publication
(10) Pub. No.: US 2005/0203348 A1
(43) Pub. Date: Sep. 15, 2005

(54) Remote cardiac arrest monitor

(76) Inventors: Musa Shihadeh, Leesburg, NJ (US); Terry Lee Torres, Leesburg, NJ (US)

Correspondence Address: ROBERT M. SKOLNIK, 353 Monmouth Road, P.O. Box 22, West Long Branch, NJ, 07764-0022, US

(21) Appl. No.: 10/994,540

(22) Filed: Nov. 22, 2004

Related U.S. Application Data
(60) Provisional application No. 60/548,780, filed on Mar. 1, 2004.

(51) Int. Cl. A61B 13/00; A61B 5/00

(52) U.S. Cl. 600/300

(57) ABSTRACT

A system for remote monitoring the human pulse rate and providing alarm indications if said pulse rate is determined to be outside predetermined high or low limits includes microcontroller means in a patient worn transmitter structure and a remote receiver structure for determining whether the pulse rate is in a dangerous zone and activating alarms indicative of such danger zone.

### CROSS REFERENCE TO RELATED APPLICATIONS

This application claims priority of provisional application 60/548,780, filed Mar. 1, 2004.

### BACKGROUND OF THE INVENTION

1. Field of the Invention

The present invention relates to automatic remote monitoring and measurement of a human's pulse rate.

2. Description of the Related Art

Vogelman, et al., U.S. Pat. No. 3,572,316, show a patient monitoring system where a number of physiological tests such as pulse and temperature are periodically sent from patients to a central station in a hospital via FM radio transmission.

Buxton, et al., U.S. Pat. No. 3,646,606, disclose another hospital FM system where EKG, blood pressure and temperature are transmitted and alarms sounded if predetermined values are reached.

Ozawa, et al., U.S. Pat. No. 4,608,994, describe a local storage system for blood pressure, etc., which transmits the measurement over phone lines or other communication link to a central station.

Ohayon, et al., U.S. Pat. No. 4,712,562, show a patient blood pressure and heart rate measurement system transmitting the information over telephone lines to a central station. Predetermined conditions for the measurement trigger additional functions.

Miwa U.S. Pat. No. 4,974,607 detects a persons EKG, etc., transmits the information over telephone lines, and detects emergency situations.

Leishman U.S. Pat. No. 5,036,852 monitors a patient for emergency conditions.

Isoyama U.S. Pat. No. 5,367,555 shows radio stations 50 in a medical monitoring system.

Stutman, et al., U.S. Pat. No. 5,416,695, shows a multi-person monitoring system connected by radio to a central station.

The published application of Eggers 2002/0186821 uses cell phone technology in a patient monitoring system.

### SUMMARY OF THE INVENTION

Coronary heart disease is one of the country's leading causes of crippling disability and/or death. Senior citizens are the most afflicted cross section of the population. Unfortunately, senior citizens are also the most vulnerable in terms of receiving I immediate emergency care following a stroke or heart attack. Many live alone, with few daily visitors, limited in their ability to reach out for immediate attention or help. Many stroke and heart attack victims fall in and out of consciousness unable to effect the world around them with the required effort to summon help. Paralyzed with pain, fear, and loss of lucidity, the simple task of reaching a phone and dialing 911 or reaching an alarm panic switch, even if worn on them as a remote devise, too often becomes a battle won by the heart disease. Each second that passes following an episode can mean the difference between life and death. It is with this concept at mind that the inventors of the R-CAM (Remote Cardiac Arrest Monitor) have designed the present invention as a system able to autonomously seek emergency assistance prior to the subject even experiencing the first sign of pain or discomfort, and without the need for the direct intervention of the subject.

The R-CAM system consists of a portable Pulse Rate Monitor Transmitter device small enough to be worn by a subject discreetly and a remotely controlled Receiver Alert Station that can be located anywhere within the dwelling place of the subject. The pulse rate monitor remotely controls the receiver alert station, which in turn can control a host of accessory alert signaling devices. R-CAM will detect the onset of a heart attack or stroke and automatically summon help without the need for the direct intervention of the subject. The receiver alert station can be interfaced with Alarm Systems, Telephone Autodialing and Message Playback Machines, Sirens etc.. With R-CAM, it is possible that before a subject even experiences the first sign of pain, the subject would be apprised and help summoned and on its way.

A principal object of the invention is the provision of a remote cardiac alarm monitor.

The foregoing, as well as further objects and advantages of the invention will become apparent to those skilled in the art from a review of the following detailed description of my invention, reference being made to the accompanying drawings.

### BRIEF DESCRIPTION OF THE DRAWINGS

FIG. 1 is a block diagram of the pulse pate monitor transmitter system;

FIG. 2 is a block diagram of the receiver alert station;

FIG. 3. is an electronic schematic diagram of the pulse ate monitor transmitter, and;

FIG. 4. is an electronic schematic of the receiver alert station.

### DETAILED DESCRIPTION OF THE PREFERRED EMBODIMENT

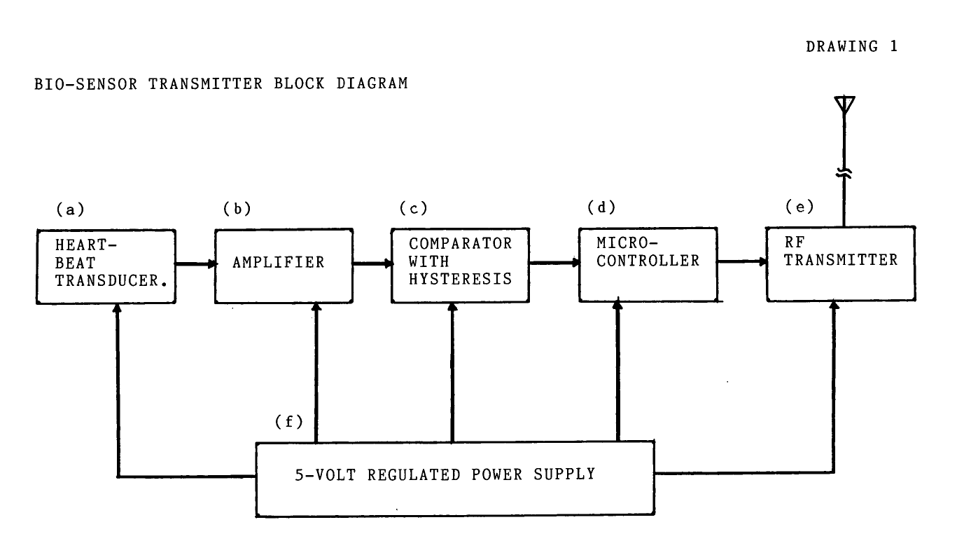

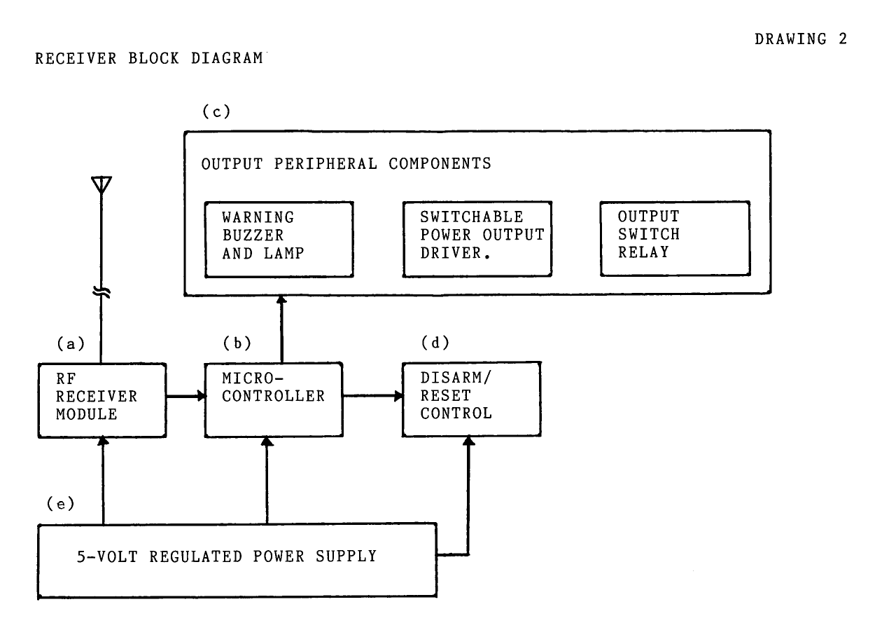

Like reference numerals have been used to designate like parts in FIGS. 1-2.

R-CAM (Remote Cardiac Arrest Monitor) technology is based upon the compilation of proven technologies to facilitate an entirely new application. All of the functional electronic building blocks that make up the entire operating R-CAM system are derived from pre-existing, tested and proven technologies. For example, the portable autonomous pulse rate monitor transmitter unit incorporates: (1) an infrared pulse rate sensor technology (heartbeat transducer) which can already be found in medical instruments; (2) a signal processor consisting of a powerful micro-controller, the full power of which is being under-exploited by the virtually trivial demands posed by the tasks required by this application; and (3) an RF transmitter module that is readily available for interface with micro-controllers and that is less than the size of a postage stamp. The combination of these three proven technologies results in a fully functional automated pulse rate monitor transmitting device that can be non-invasively worn by a subject.

Likewise, the Receiver Alert Station is also an evolution of similarly proven technologies.

The R-CAM system is comprised of two separate electronic devices, which work together. These devices are described below:

Pulse Rate Monitor Transmitter Unit:

The first device is a light weight, portable, hook and loop fastened, band strapped bracelet styled pulse rate monitor that can be discreetly worn about the extremities. The pulse rate monitor transmitter unit uses an infrared emitting diode and a phototransistor in a reflective photo-sensor configuration to bounce a low intensity infrared beam off the surface of the subject's skin, detecting the small variations in luminosity as the skin's reflectivity changes in direct response to the changing density of blood flow (as the heart beats). The pulse rate monitor transmitter unit device continuously monitors the pulse rate of the subject. If the subject's pulse rate should increase or decrease beyond levels deemed within the normal range of cardiac activity, the pulse rate monitor's micro-controller generates a binary security code for encoding by the transmitter to propagate a panic alert signal via an RF link to a receiver alert signaling device.

Receiver, Alert Signaling Device:

The second component is an RF Receiver with a micro-controller signal processor capable of demodulating and recognizing the proper system's activation security code. The Receiver features consist of a built in alert signaling buzzer with 10-second (optional) to full system activation delay, latching relay switch output (universal control interface), built in alert lamp, and switchable power output (interface). The Receiver Alert Station may be directly connected to an alarm system, automated telephone dialing and message playback machine, lights, sirens, or any combination of the above components or devices. When in use, the system continuously awaits a valid RF distress signal from the subject's pulse rate monitor transmitter unit. This state is referred to as the systems stand by mode. When it receives a valid RF distress signal from the pulse rate monitor transmitter unit the system activates its internal warning features sounding a pulsating piezo buzzer and flashing an LED lamp for a period of 10-seconds (optional). If the system is not manually reset by the elapse of the 10-second warning period the receiver alert station latches its output interfaces in an ON state; instantly triggering whatever alert signaling device was chosen for interface (i.e. alarm systems, telephone auto dialer, etc.).

Operating Mechanism:

The pulse rate of a healthy individual may be affected by many factors. Generally however, heart rate variations of a healthy individual can be within a range of 60 to 180 beats per minute (bpm). However, when an individual experiences a stroke or heart attack their pulse rate will typically fall below 60 bpm or rise above 180 bpm. The R-CAM Bio-Transmitter's signal processor is designed to alert to the breach of these threshold limits. However, it should be noted that the above 60/180 bpm threshold is solely a general case and the need for deviance from these parameters may be required due to special needs, subject age, or special circumstances. The processor threshold limits are subsequently made programmable variable to meet the consumer's specific needs.

Block Diagram:

FIG. 1 is a block diagram of the R-CAM transmitter system. The system of the pulse rate monitor transmitter unit includes (a) a Heartbeat Transducer; (b) Amplifier; (c) Voltage Comparator with Hysteresis; (d) Micro-controller; (e) an RF Transmitter; and (f) Power Supply. (a) The Heartbeat Transducer detects a subject's pulse and converts the pulse rhythm into corresponding electrical pulses which are directly coupled to the input of a standard Amplifier (Block b). The Amplifier b increases the Heartbeat Transducer's signal strength and directly couples the amplified signal into the input of a standard Voltage Comparator with Hysteresis (Block c).The Voltage Comparator with Hysteresis c generates a pulsed high (or low) signal output in response to every beat of the subject's pulse. The output of the voltage comparator c is coupled directly into the input of the micro-controller (Block d) described above for purposes of signal processing.

Block assemblies (a), (b), and (c) above, are what are termed the Bio-Sensor Detector circuitry. Working together these assemblies detect, amplify, and produce a pulsed high (or low) square wave signal output that is in direct accordance with the subject's pulse rate. Each time the subject's heart beats the output of this overall assembly pulses from Low to High (or high to Low). The time period pulsed high (or low) and the quiescent time period remaining low (or high) are variably proportional to the subject's cardiac cycle. These factors also contain vital information, which may optionally be drawn upon to fulfill special application analysis.

The Micro-controller d serves as the decision making and control device. The micro-controller performs signal processing on the inputted signal originating from the output of the Bio-Sensor Detector circuitry. In a basic application the micro-controller may count the number of pulses, which occur at its input over a period of 1-minute to ascertain the subject's pulse rate. Next, it may compare this value against two programmed values, one of which is the lowest value acceptable (60 bpm) and the other of which is the high value acceptable (180). If the measured value falls between the two limiting values the micro-controller would take no further action. Another sampling/processing cycle would be initiated immediately after the conclusion of every uneventful sampling/processing cycle. If the measured value falls outside of either of the two limiting values, the micro-controller would enable the RF Transmitter and encode the propagated RF wave with a security code recognizable to the receiver alert station as being an alert activation command. The micro-controller may repeat this RF transmission repeatedly over short intervals until the unit is manually reset.

(e) The RF Transmitter Module e is a commercially available device designed to facilitate wireless Micro-controller communication links. The RF Transmitter encodes the signal applied to it from the micro-controller using AM or FM encoding principles and propagates the modulated RF signal when enabled to do so by the micro-controller. The RF transmitter module may (arbitrary) operate within the 310 or 900 Mhz Band.

(f) The Power Supply f is simply the unit's power source. The unit may be designed to operate on as little as 3 volts or as high as 9 volts. Batteries of these specifications are readily available on the consumer market.

Receiver Alert Station:

FIG. 2 is a block diagram of an individual Receiver Alert Station. It includes: (a) RF Receiver Module; (b) Micro-controller; (c) Output Control Interface Components; (d) Disarm/Reset Control; and (e) Power Supply.

Circuit Block Description:(a) The RF Receiver Module a is a commercially available device designed to facilitate wireless Micro-controller communication links. The module receives and demodulates the RF carrier wave propagated by the matching RF Transmitter module. The module provides the actual binary intelligence encoded upon the carrier wave, to the input of the proceeding micro-controller.

(b) The Micro-controller in this application simply serves as the RF Receiver Module's serial data transmission processor and output initiation control device. The Micro-controller essentially awaits an RF transmission from the pulse rate monitor transmitter unit (bearing the proper alert activation code), to respond by opening/closing universal interface relays, switch On auxiliary power output interfaces, and drive internal warning buzzers and lamps.

(c) The Output Control Interface Components are the actual components used to facilitate auxiliary control over external systems and devices. These components can be as simple as the common relay used to trigger alarm systems or they may be power transistors used to power external devices.

(d) The Disarm/Reset Control is a push button switch, which can disarm and reset the unit at any time it is pressed.

(e) The Power Supply is the unit's power source. Since the receiver alert station can be located anywhere within the dwelling place of the subject, the unit may be powered by a standard 12-volt regulated wall transformer power supply.

Micro-Controller Software:

The instruction set of the software used in this system gives this technology its personality. As with any complex system, custom software must be written to meet the particular needs of the application for which it will be used. Whereby, the particular software solution used for a particular application will vary from the needs of one application to that of another. No one software program can be written to meet the innumerable needs under which this application will operate. Micro controllers, input/output (I/O) interface hardware, and their software solutions are not new inventions requiring a detailed explanation as to their operation or feasibility. Further, the power of their processing capabilities is clearly well beyond the trivial demands posed by this application. As a result, a specific instruction set for micro-controller use shall not be specified herein, and the instruction set shall be referred to herein in generically descriptive terms. The software will always incorporate common command structures.

Pulse Rate Monitor Micro-Controller Software:

A generalized application uses a program that instructs the micro-controller to begin a 1-minute timing interval and count the number of individual pulses generated by the Bio-Sensor Detector circuitry present at one of the micro-controller's input pins. Upon the elapse of the 1-minute timing mark the software would instruct the micro-controller to stop counting and store the counted value for subsequent comparison purposes. The software would now instruct the micro-controller to compare the actual counted value against a fixed low programmed value (60 bpm) and a fixed high programmed value (180 bpm) and generate a caution flag only if the actual counted value is found to be less than the low programmed value or above the high programmed value. The software would next instruct the micro-controller to check an assigned register for the presence of a caution flag. If a caution flag is present, the software instructs the micro-controller to power the RF Transmitter module and transmit an encoded alert activation code. The software may instruct the micro-controller to continuously re-transmit this distress signal in short intervals until such time as the unit is manually shut down. However, if no caution flag exists the micro-controller would conclude this sampling cycle and reset. The software would then initiate the repeated commencement of new sampling/processing cycles, checking for caution flags upon completion of each cycle.

Receiver Alert Station Micro-Controller Software:

A generalized application may simply use a program that instructs the micro-controller to poll the input pin coupled to the RF Receiver Module for the presence of a serial formatted start bit. The software would instruct the micro-controller to do nothing until a start bit is received. At this time the output interfaces are quiescent and all alert signaling devices inactive. When a start bit is polled at the input pin of the micro-controller, the software would instruct the micro-controller to begin shifting in the serial data bit string received. Upon completion of the transmission, the software would instruct the micro-controller to compare the data string value received against a fixed value stored in memory. If the received value is equal to the stored value, the software would activate a 10-second (optional) warning buzzer that alerts inhabitants that a full alert activation state will be initiated if the system is not immediately manually reset. If after 10-seconds (optional) the system is not manually reset, the software will instruct the micro-controller to power all peripheral components (interfaced to the micro-controller's Input/output pins) in a latched On condition. This state would persist until a manually activated reset command was initiated. However, if the serial data bit-string received does not equal the fixed value stored in memory, the software would instruct the micro-controller to reset and await the reception of the next start bit, This cycle would continue indefinitely.

Pulse Rate Monitor Transmitter, Schematic:

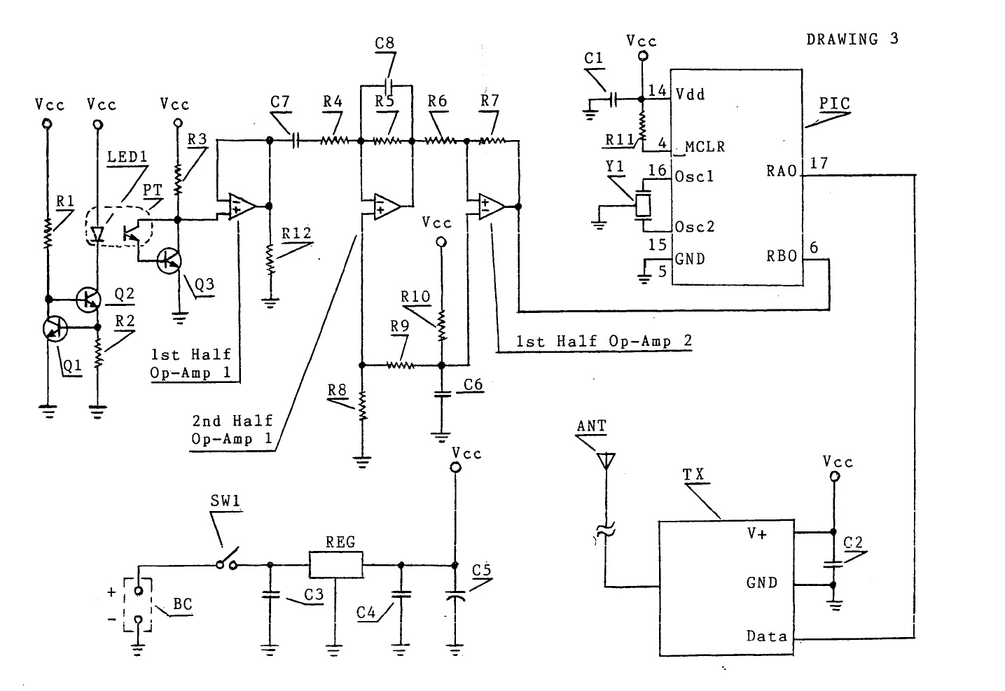

FIG. 3 is a schematic diagram of the pulse rate monitor transmitter of FIG. 1.

Component Description:

1. Component BC, SW1, REG, C3, C4, and C5, form the unit's power supply circuitry. BC is a 9-volt battery clip, SW1 is the On/Off switch, REG is a 5-volt regulator and components C3, C4, and C5 are the regulator's filtering capacitors.

2. Component LED1 is an infrared emitting diode. Components Q1, Q2, R1, R2 and LED1 form the Infrared LED driver circuit which features a constant current source configuration which aids in facilitating a constant luminous output. Components LED1 and PT form a reflective infrared photosensor.

3. Component PT is an infrared phototransistor. Components PT, Q3, and R3 form a high gain Darlington configured infrared amplifier. The output of this amplifier is directly proportionate to the intensity of the infrared light to which it is exposed. Components LED1 and PT form a reflective infrared photosensor.

4. Components 1st half Op-Amp 1, and R12 form a conventional buffer amplifier which isolates the output load of the Darlington configured infrared amplifier and provide drive to the proceeding amplifier stage.

5. Component C7 is an input coupling capacitor which blocks DC voltages at the amplifiers input terminal. Also creates a high-pass filter with component R4 at: fc=1/(2×3.14×C7×R4).

6. Component R4, R5, C8 and 2nd half Op-Amp 1 form an inverting amplifier. The gain of this amplifier is set at: Av=(R5/R4) Components R5 and C8 also create a low-pass filter which bandwidth limits the amplifier and prevents high frequency oscillation bursts. Fc=1/(2×3.14×C8×R5).

7. Components R6, R7 and 1st half Op-Amp 2 form a Comparator with Hysteresis.

8. Components R8, R9, and R10 form a resistive bias string for the amplifier comprised of by the 2nd half Op-Amp 1 and the Comparator comprised of by the 1st half Op-Amp 2. This bias string biases the output of the amplifier below the trigger threshold of the comparator forcing the output of the Comparator low in its quiescent state, and of which is switch high momentarily during active pulse detection.

9. Components PIC1, Y1, R11, and C1 form the micro-controller circuitry. The micro-controller is responsible for signal processing.

10. Components TX and C2 are the RF Transmitter module and power supply bypass capacitor. The module herein described is an AM modulated transmitter that pulses its propagated carrier wave ON and OFF in direct accordance with the digital state inputted to its DATA pin.

Circuit Operation Description:

The operation of the pulse rate monitor transmitter circuit is as follows. SW1 is the units On/Off Switch. By closing this switch 5-volt regulated power will be applied throughout the circuit, sourced from a standard 9-volt Nickel Cadmium Rechargeable Battery. The circuit will be energized and the micro-controller will begin to execute its program.

Components R1, R2, Q, Q2, LED1 and PT, Q3, R3 form a reflective -infrared heartbeat transducer assembly. The Heartbeat Transducer uses changes in the skins reflectivity caused by blood density changes produced by the subjects heartbeat to modulate the reflected source of Infrared light that is detected by a phototransistor implemented in a Darlington amplifier configuration. The phototransistor/amplifier circuit converts these light fluctuations into corresponding voltage variations. Whereby, when the heart beats, the blood density of the skin enhances reflectivity and increases the intensity of the reflected light source and the quiescent voltage appearing at the collector of Q3 swings low in response. After a brief moment the blood density dissipates, the skin's reflectivity decreases, and the signal voltage appearing at the collector of Q3 returns to its higher quiescent value. In summary, each time the heart beats the voltage at the collector of Q3 swings low and as the blood density dissipates between beats the voltage at the collector of Q3 returns to its quiescent value.

Moments after the subject's heart beats, the heartbeat transducer detects the skins enhanced reflectivity and the voltage at the collector of Q3 swings low. The buffer amplifier consisting of the composition surrounding 1 st half Op-Amp 1 follows the voltage swing and couples the falling signal into the input of the inverting amplifier consisting of the composition surrounding 2nd half Op-Amp 1. The output of the amplifier is biased below the trigger threshold of the proceeding Comparator state and holds the output of the Comparator low in its quiescent state. The signal is amplified and the output of the amplifier swings high tripping the threshold of the Comparator and in return forcing the comparator's output to switch high abruptly. The blood dissipates shortly after the initial thrust of the cardiac output and the skin's reflectivity begins to lessen. The voltage at the collector of Q3 begins to rise toward its quiescent level. The amplifier output begins to fall below the trip threshold of the comparator stage. The Comparator output abruptly returns to its quiescent low state. These events take place each time the subject's heart beats. In summary, each time the subject's heart beats the output of the comparator abruptly swings high until the blood density dissipates prior to the proceeding cardiac output (heart beat) and forces the output of the comparator to return to its quiescent low state. Whereby, the process repeats itself with every heart beat.

The micro-controller begins a timing interval of know duration. At the commencement of this timing interval the micro-controller enables its input RB0 pin and begins to poll pin ¶6 for a change of Comparator output state. When the output state of the Comparator switches high, the micro-controller detects the change of state and assigns the event a value of one. This value is stored in a register. Each subsequent change of state from low to high is likewise detected and assigned a value of 1 and added to the previous value stored in register and the product of these additions are returned to register. This process is equivalent to counting the number of events and storing the total. Upon the elapse of this time interval the final value stored in register is compared against a low and high value stored in memory. These low and high values are the programmed values representative of normal cardiac activity with respect to the timing interval implemented. If the measured value is found to be below or above the low and high programmed values stored in program memory the micro-controller will shift out an 8-bit binary security code sequences through RA0 pin ¶17 interfaced to the RF Transmitter Module enable/disable pin labeled: DATA. Whereby, the RF Transmitter will propagate a carrier wave encode with the intelligence of the 8-bit binary security code sequence. However, if the measured value is found to be between the low and high programmed values stored in memory the microcontroller will reset and begin a new sampling cycle. The process repeats after each uneventful cycle—indefinitely.

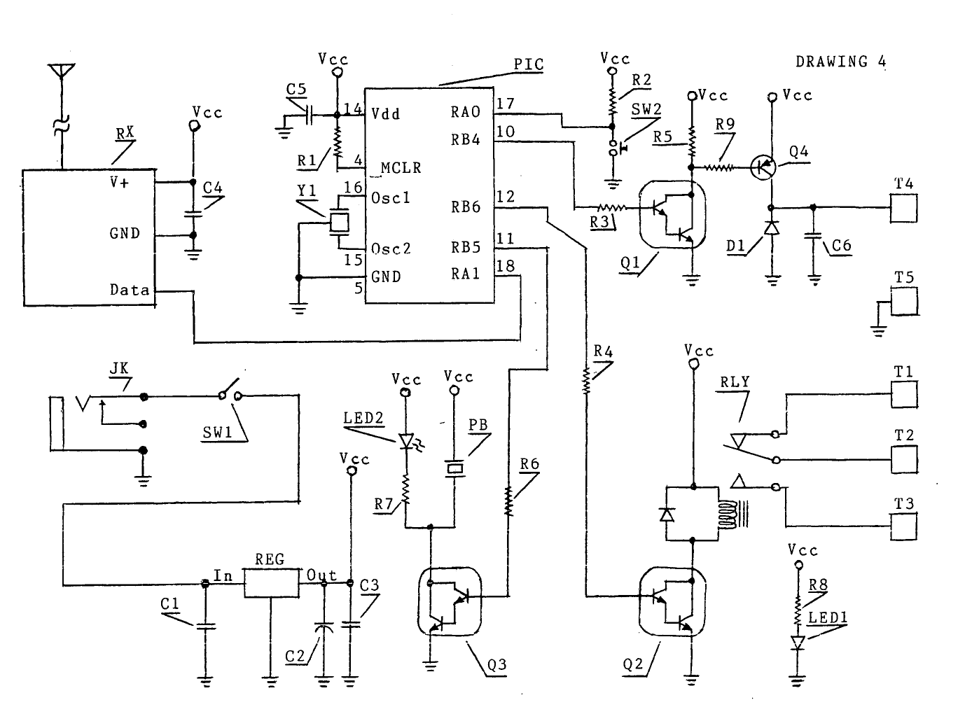

Receiver Alert Station, schematic: FIG. 4 is a schematic diagram of the Receiver Alert Station.

Component Description:

1. JK, SW1, REG, C1, C2, C3, R8, and LED1 form the unit's 5 Volt power supply control circuitry and power status indicator lamp. This device is powered by a standard 12-volt wall transformer.

2. Components RX, C4, and ANT consists of a commercially available RF Receiver Module compatible with Microcontroller interface. This device incorporates all of the RF detection, amplification, heterodyne, and signal processing hardware required to decode the intelligence carried within the RF carrier wave and reproduce the actual sequential binary format encoded at the transmitter.

3. Components PIC2, Y1, C5, R1, R2, and SW2 form the Receiver Alert Station's Microcontroller decision making and control circuitry.

4. Q3, R6, R7, LED2, and PB form the receiver's internal warning alert indicators consisting of a 98 dB Piezo Buzzer and an LED indicator lamp.

5. Components Q1, Q4, D1, R3, R5, R9, and C6 form the receivers switchable auxiliary power output, capable of driving external loads of up to 100 mA at 5 volts.

6. Components Q2, R4, and RLY form the receiver's auxiliary output switch capable of controlling externally interfaced loads and/or triggering alarm systems, telephone dialing and automated message playback machines, etc.

Circuit Operation Description: The operation of the Receiver Alert Station circuit shown in FIG. 4 is as follows: SW1 is the unit's On/Off Switch. By closing this switch 5-volt regulated power will be distributed throughout the circuit. When power is distributed throughout the entire circuit, LED1 will illuminate. The circuit will be energized and the microcontroller will begin to execute its program.

The RF Receiver Module, RX, awaits signaling from the pulse rate monitor transmitter. In this quiescent state the receiver's output remains in a low state (equivalent to binary 0). When the receiver detects active signaling from the Transmitter it automatically demodulates the carrier wave and extracts the binary security code sequence encoded upon the wave and presents this intelligence in serial format to the output pin of the device which is coupled to the input of the microcontroller through pin ¶18 (RA1). A binary 1 would be represented by the output swinging high and a binary 0 represented by a low state output.

The 10-second Warning Delay Signaling feature (explained below is comprised of components Q3, R6, R7, PB and LED2. When output pin 11 (RB5) is in a low state, no current can flow into the base of the Darlington transistor Q3, essentially keeping the switch in an Off state. Whereby, the piezo buzzer PB and LED2 remain in a cut Off state. When output pin 11 (RB5) is caused by the microcontroller to switch to a high state, current begins to flow into the base of the Darlington transistor Q3, essentially switching the switch completely On and driving the collector to a low state, whereby, the piezo buzzer PB and LED2 begin to sound and illuminate as power is applied to both which are in parallel with each other and in series with the collector to the positive supply. If the output pin is caused to switch On and Off once or twice a second, the piezo buzzer would begin to generate a pulsating tone and LED2 would be seen to flash.

The Auxiliary Power Output is comprised of components Q1, Q4, R3, R5, R9, D1, C6, T4 and T5. When output pin 10 (RB4) is in a low state, no current can flow into the base of the Darlington transistor Q1, essentially keeping the switch in an Off state, whereby, the collector of Q1 remains at the positive supply voltage which in turn keep the base of Q4 unbiased. The unbiased state of Q4 disables the collector and no voltage appears at the terminals of the Auxiliary Power Output (across Interface Terminals T4 and T5). When output pin 10 (RB4) is switched to a high state by the microcontroller, current begins to flow into the base of the Darlington transistor Q1, essentially switching the switch full On. Whereby, the collector of Q1 is driven into a low state biasing the base of Q4 through resistor R9. Current begins to flow into the base of Q4 essentially switching the switch hard On and driving the collector to a high state. The biased state of Q4 enables the collector and the full power supply voltage (minus the transistors saturation voltage) appears across the terminals of the Auxiliary Power Output (Interface Terminals T4 and T5). When the Microcontroller switches the output from high to low, the power appearing across the terminals of the Auxiliary Power Output disappears, and power is cut Off.

The Auxiliary Output Control Switch is comprised of components Q2, R4, RLY, and Interface terminals T1, T2, and T3. When output pin 12 (RB6) is in a low state, no current can flow into the base of the Darlington transistor Q2, essentially keeping the switch in an Off state, whereby, the Relay switch RLY remains in a cut Off state. Terminals T2 and T1 remain in a closed state and terminals T2 and T3 remain in an open state. When output pin 12 (RB6) is caused by the microcontroller to switch to a high state, current begins to flow into the base of the darlington transistor Q2, essentially switching the switch hard On and driving the collector to a low state. Whereby, full supply power is placed directly across the Relay switch, RLY, forcing the contacts to close. Terminals T2 and T1 switch to an open state and terminals T2 and T3 switch to a closed state.

At start up, Microcontroller PIC2 continuously polls the input sourced by the output of the RF Receiver Module. When the input pin (pin ¶18 RA1) is polled and found not to contain an active high start bit, the systems output alert features remain inactive. When the microcontroller polls the input pin and detects a start bit, represented by the output of the RF Module output switching to a high state, the system begins to shift in the binary intelligence at the programmed transmission rate. After shifting in the binary intelligence, the microcontroller compares the binary value of the received transmission against a programmed binary security code quantity stored in program memory. If the received binary value is not equal to the stored security code quantity, than the microcontroller disregards this transmission as noise, resets, begins to re-poll the input line continuously, and takes no further action. However, if the received value is equal to the stored security code quantity, than the microcontroller activates the units internal 10-second (optional) warning delay (with respect to full system activation).

The 10-second (optional) warning delay consisting of the activation of the unit's internal piezo buzzer PB and illumination of lamp LED2. The microcontroller initiates this feature by switching output pin ¶11, RB5, to a high state. Next, during the 10-second (optional) warning delay period the microcontroller begins to poll pin ¶17 (RA0) which functionally serves as an active systems reset input. If switch SW2 is pressed any time during the 10-second warning delay period, the system will reset and the microcontroller will begin to poll the input from the Receiver Module for another transmission containing the proper security code sequence, and no further action shall be taken by the microcontroller. However, if switch SW2 is not pressed by the elapse of the 10-second warning delay interval, the microcontroller will activate both the Auxiliary Power Output (Q4), and the Auxiliary Output Switch (RLY), instantly triggering whatever alert devices were interfaced by these control ports. The microcontroller initiates these features by simultaneously switching pins ¶10 (RB4) and ¶18 (RB6) to a high state.

Cosmetic Packaging:

The Pulse Rate Monitor Transmitter assembly can be packaged within a standard ABS plastic enclosure with a built in 9-volt battery compartment of 79 mm×57 mm×23 mm dimensions. The unit may be secured about the subject's lower leg (or alternative extremity), by the use of a 79 mm wide elastic-band-strap having parted ends that are secured by a Hook and Loop fastener system to hold the enclosure beneath the band's tension. This will allow a stable carrier medium which facilitates the means by which to have direct reflective photodetection of the subjects skin surface through the photosensor module's reflective beam port being internally positioned above a hole machined through the enclosures surface to be oriented skin contact side down. The enclosure's texture, color, and the exact enclosure positioning of the On/Off switch (the only user control) is not critical and optional. The Receiver Alert Station can be packaged within a standard table-top ABS plastic enclosure of 120 mm×90 mm×30 mm dimensions. The enclosures texture, color, physical positioning of the units On/Off switch, Reset Switch, Piezo Buzzers sound escape hole, LED1, LED2, Power Jack, Antenna, and Interface Block Terminals are not critical and are all optional.

A list of the components of FIGS. 2 and 4 are provided on the following pages.

### PARTS IDENTIFIER LIST

R-CAM Bio-Transmitter.

The following parts may be purchased from the following source:

DIGI-KEY

701 Brooks Ave. South P.O. Box 677 Thief River Falls, Minn. 56701-0677 1-800-344-4539

| Sym. | Part No. | Description |
|---|---|---|
| OP-AMP1 | LM358AM-ND | Low Power Dual Op-Amp |
| OP-AMP2 | LM358AM-ND | Low Power Dual Op-Amp |
| PIC | PIC16F84-04I/SO-ND | 8-Bit CMOS Microcontroller |
| TX | TX-66-ND | RF Transmitter Board 310 MHz |
| Y1 | X902-ND | 4 Mhz Ceramic Resonator |
| LED1 | 160-1028-ND | Infrared Diode Vf = 1.2 If = 50 mA |
| PT | 160-1030-ND | Photo Transistor |
| REG | LM2931AZ-5.0-ND | 5 Volt Positive Regulator |
| Q1 | 2N3904-ND | NPN Transistor (2N3904SM) |
| Q2 | 2N3904-ND | NPN Transistor (2N3904SM) |
| Q3 | 2N3904-ND | NPN Transistor (2N3904SM) |
| R1 | P-10K-GCT-ND | 10K Ohm 5% Chip Resistors |
| R2 | P-130-GCT-ND | 130 Ohm 5% Chip Resistors |
| R3 | P-10K-GCT-ND | 10K Ohm 5% Chip Resistors |
| R4 | P-1.0K-GCT-ND | 1.0K Ohm 5% Chip Resistors |
| R5 | P-1.0M-GCT-ND | 1.0M Ohm 5% Chip Resistors |
| R6 | P-1.0K-GCT-ND | 1.0K Ohm 5% Chip Resistors |
| R7 | P-1.0M-GCT-ND | 1.0M Ohm 5% Chip Resistors |
| R8 | P-1.0M-GCT-ND | 1.0M Ohm 5% Chip Resistors |
| R9 | P-100K-GCT-ND | 100K Ohm 5% Chip Resistors |
| R10 | P-1.0M-GCT-ND | 1.0M Ohm 5% Chip Resistors |
| R11 | P-10K-GCT-ND | 10K Ohm 5% Chip Resistors |
| R12 | P-10K-GCT-ND | 10K Ohm 5% Chip Resistors |
| C1 | PCF1046CT-ND | .01 Microfarad Film Capacitor |
| C2 | PCF1046CT-ND | .01 Microfarad Film Capacitor |
| C3 | PCF1046CT-ND | .01 Microfarad Film Capacitor |
| C4 | PCF1046CT-ND | .01 Microfarad Film Capacitor |
| C5 | P5578-ND | 10 Microfarad Electro. Cap. |
| C6 | PCF1046CT-ND | .01 Microfarad Film Capacitor |
| C7 | P1168-ND | 220 Microfarad Bi-Polar Cap. |
| C8 | PCF1012CT-ND | .001 Microfarad Film Capacitor |
| SW1 | EG1847-ND | Right Angle PC Mount Slide Switch |
| BC | | 9-Volt Battery Contacts |
| ENC | SRM6A-ND | M6 Series Plastic Enclosure |

### PARTS IDENTIFIER LIST

R-CAM Receiver Unit.

The following parts may be purchased from the following source:

DIGI-KEY

701 Brooks Ave. South P.O. Box 677 Thief River Falls, Minn. 56701-0677 1-800-344-4539

| Sym. | Part No. | Description |
|---|---|---|
| RX | RE-n6-ND | RF Receiver Board 3110 MHz |
| PIC | PIC16F84-04I/ | 8-Bit CMOS Microcontroller |
| | SO-ND | |
| Y1 | X902-ND | 4 Mhz Ceramic Resonator |
| REG | LM2931AZ-5.0-ND | 5 Volt Positive Regulator |
| LED1 | 160-1144-ND | Green Colored LED T-1 2.1 20 mA |
| LED2 | 160-1139-ND | Red Colored LED T-1 2.1 20 mA |
| D1 | 1N5817DICT-ND | Schottky Barrier Rectifier |
| Q1 | MPSA14-ND | NPN Darlington Transistor |
| Q2 | MPSA14-ND | NPN Darlington Transistor |
| Q3 | MPSA14-ND | NPN Darlington Transistor |
| Q4 | MPSA14-ND | NPN Darlington Transistor |
| R1 | 10KQBK-ND | 10k Resistor 5% Carbon |
| R2 | 10KQBK-ND | 10k Resistor 5% Carbon |
| R3 | 10KQBK-ND | 10k Resistor 5% Carbon |
| R4 | 10KQBK-ND | 10k Resistor 5% Carbon |
| R5 | 10KQBK-ND | 10k Resistor 5% Carbon |
| R6 | 10KQBK-ND | 10k Resistor 5% Carbon |
| R7 | 100QBK-ND | 100 Resistor 5% Carbon |
| R8 | 130QBK-ND | 130 Resistor 5% Carbon |
| R9 | 130QBK-ND | 4.7K Resistor 5% Carbon |
| C1 | P3488-ND | 0.10 Microfarad Polypropylene Cap |
| C2 | P3488-ND | 0.10 Microfarad Polypropylene Cap |
| C3 | P5517-ND | 100 Microfarad Electrolytic Cap |
| C4 | P3488-ND | 0.10 Microfarad Polypropylene Cap |
| C5 | P3488-ND | 0.10 Microfarad Polypropylene Cap |
| C6 | P3488-ND | 0.10 Microfarad Polypropylene Cap |
| RLY | HE112-ND | SPDT Relay |
| JK | CP-002APJ-ND | Male Panel Mount Power Jack |
| ENC2 | SR031A-ND | 82.55 × 111.25 × 22.86 mm Enclosure |
| T1-5 | CBB102-ND | 2 Contact Barrier Block |
| PB | P9948-ND | Piezo Audio Signal Device |
| SW1 | CKN1189-ND | Push Button Toggle Switch |
| SW2 | CKN1189-ND | Push Button Momentary Switch |
| WT | T506-ND | AC-DC Wall Tranormer 12n-Volt 500 mA |
| ANT | | |

Further modifications to the invention may be made without departing from the spirit and scope of the invention; accordingly, what is sought to be protected is set forth in the appended claims.

1. A remote cardiac arrest monitor comprising: a transmitter including pulse rate sensing means for producing an electrical signal representing a human pulse rate; amplifier means connected to said pulse rate sensing means for amplifying said electrical signal; comparator means with hysteresis connected to said amplifier means for generating a pulsed output signal representing each beat of said human pulse; transmitter microcontroller means connected to said comparator means for determining the pulse rate of said human pulse and for comparing said pulse rate against predetermined high and low values for said pulse rate and for generating an output signal if said pulse rate is outside said predetermined values; radio frequency transmitter means connected to said microcontroller means for transmitting said microcontroller means output signal to a remote station; power supply means connected to supply electrical power to said transmitter; and means at said remote station for providing an alarm indication.

2. The remote cardiac arrest monitor of claim 1 wherein said remote station alarm indication mean includes radio frequency receiver means for receiving said transmitter microcontroller means output signal transmitted by said transmitting means; receiver microcontroller means connected to said receiver means for generating an output alarm control signal; a plurality of output alarms connected to said microcontroller means for providing an alarm indication; disarm and reset control means connected to said microcontroller means for disabling said alarms; and power supply means connected to supply electrical power to said remote station alarm indication means

3. A remote cardiac arrest monitor comprising a transmitting station being removeably attached to a patient and a receiving station remote from said transmitting station for providing alarm indications if the patient's pulse rate is outside predetermined limits; said transmitting station including pulse rate determining means coupled to the patient's skin for providing an electrical output signal representing the patient's pulse rate; amplifier means attached to said pulse rate determining means for amplifying said electrical output signal; comparator means attached to said amplifier means for converting the output of said amplifier means to a pulse rate signal; microcontroller means connected to said comparator means for generating an output signal if said pulse rate signal is outside predetermined high and low limits; transmitter means connected to said microcontroller means for transmitting said output signal to said receiving station; receiver means at said receiver station for receiving said transmitted signal; microcontroller means connected to said receiver means for generating an output alarm signal; and alarm indication means connected to said microcontroller means for providing visual and audible alarm indications at said receiving station.
